(19) United States
(12) Patent Application Publication
(10) Pub. No.: US 2019/0224502 A1
(43) Pub. Date: Jul. 25, 2019

(54) Method for Sunless Tanning and Method for Darkening White Hair

(71) Applicant: Yu-Jen Wu, Kaohsiung City (TW)

(72) Inventors: Yu-Jen Wu, Kaohsiung City (TW); Tzu-Yang Lin, Kaohsiung City (TW)

(21) Appl. No.: 16/199,323

(22) Filed: Nov. 26, 2018

(30) Foreign Application Priority Data
Jan. 22, 2018 (TW) 107102227

(51) Int. Cl. A61Q 19/04; A61K 8/34; A61K 8/64

(52) U.S. Cl. CPC A61Q 19/04; A61K 8/8164; A61K 8/64; A61K 8/34

(57) ABSTRACT

A method for sunless tanning is disclosed. The method includes applying an effective amount of an extract of cocoa rind to a skin of a subject in need thereof. The extract of cocoa rind is obtained by extracting a sample of cocoa rind by an aqueous ethanol solution with a concentration of ethanol being 50-75%. A method for darkening white hair is also disclosed.

### CROSS REFERENCE TO RELATED APPLICATIONS

The application claims the benefit of Taiwan application serial No. 107102227, filed Jan. 22, 2018, the entire contents of which are incorporated herein by reference.

### BACKGROUND OF THE INVENTION

### 1. Field of the Invention

The present invention generally relates to a method for sunless tanning and, more particularly, to a method for sunless tanning using an extract of cocoa rind. The present invention also relates to a method for darkening white hair using the extract of cocoa rind.

### 2. Description of the Related Art

People can get a beautiful tan not only by sun tanning, but also by applying a tanning agent to the skin. Conventional tanning agents generally include dihydroxyacetone as an active ingredient. When the conventional tanning agents are applied to skin of a user, dihydroxyacetone combines to arginine in epidermal cells. Therefore, the skin of the user turns brown, so called a brown artificial tan.

However, dihydroxyacetone has an unpleasant odor, and the brown artificial tan can only last for a week. Moreover, the skin applied with the conventional tanning agents including dihydroxyacetone turns orange (orange tan) if the skin contacts with acid substances. In light of this, it is necessary to provide a method for sunless tanning.

### SUMMARY OF THE INVENTION

It is therefore an objective of the present invention to provide a method for sunless tanning using an extract of cocoa rind.

It is another objective of the present invention to provide a method for darkening white hair using the extract of cocoa rind.

An embodiment of the present invention discloses a method for sunless tanning. The method includes applying an effective amount of an extract of cocoa rind to a skin of a subject in need thereof. The extract of cocoa rind is obtained by extracting a sample of cocoa rind by an aqueous ethanol solution with a concentration of ethanol being 50-75%. As an example, the extract of cocoa rind is applied in the effective amount being 200-400 mg/cm².

Another embodiment of the present invention discloses a method for darkening white hair. The method includes applying an effective amount of an extract of cocoa rind to a white hair of a subject in need thereof. The extract of cocoa rind is obtained by extracting a sample of cocoa rind by an aqueous ethanol solution with a concentration of ethanol being 50-75%. As an example, the extract of cocoa rind is applied in the effective amount being 200-400 mg/cm².

Accordingly, the active ingredients extracted from the sample of cocoa rind can effectively increase the expression level and the activity of tyrosinase in melanocytes, improving the melanin level in melanocytes. Therefore, the extract of cocoa rind can be used to applied for manufacturing a tanning agent or a black hair agent. As such, the cocoa rind, which is usually thought as the waste of manufacturing chocolate, has a new economic output.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 depicts a bar chart showing relative survival rates of the B16-F10 cells of groups A0-A3.

FIGS. 2a-2d depict micrographs showing cell numbers of the B16-F10 cells of groups A0-A3, respectively.

FIG. 3 depicts a bar chart showing relative melanin levels of the B16-F10 cells of groups B0-B3.

FIG. 4a depicts a Western blot showing tyrosinase activity of the B16-F10 cells of groups C0-C3.

FIG. 4b depicts a bar chart showing relative band intensity of FIG. 4a.

FIG. 5 depicts Western blots showing levels of proteins associated with melanin synthesis, such as tyrosinase, TRP-1 and DCT, in the B16-F10 cells of groups D0-D3.

### DETAILED DESCRIPTION OF THE INVENTION

The term “agent” refers to a preparation including a predetermined amount of an active substance in a specific formulation. Moreover, the term “tanning agent”, as well as the term “black hair agent”, refers to the agent which increases the expression level and the activity of tyrosinase in melanocytes, improving the melanin level in melanocytes, which can be appreciated by a person having ordinary skill in the art. Detail description is not given to avoid redundancy.

The term “cocoa rind” refers to a shell left after cocoa beans are drawn out from a cocoa pod. Specifically, the cocoa beans drawing out from the cocoa pod can be applied to manufacture chocolate after fermentation, solarization and roasting; and therefore, the cocoa rind is usually thought as a waste of manufacturing chocolate.

An extract of cocoa rind according to an embodiment of the present invention can refer to a product obtained by extracting a sample of cocoa rind by an aqueous ethanol solution used as an extractant. As an example, a worker can mix the aqueous ethanol solution (100-300 mL, with the concentration of ethanol being 50-75%) with the sample of cocoa rind (100 grams) After extracting by the extractant at 60-80° C. for 6-8 hours, a supernatant is obtained by centrifugation at 8,000-12,000 rpm for about 30-45 minutes. The supernatant is then filtered, concentrated at a reduced pressure, and frozen dried to form the extract of cocoa rind.

The extract of cocoa rind according to the embodiment of the present invention can increases the expression level and the activity of tyrosinase in melanocytes, improving the melanin level in melanocytes. Therefore, the extract of cocoa rind can be used to applied for manufacturing a tanning agent or a black hair agent. The extract of cocoa rind can be used individually, or in combination with pharmaceutical acceptable vehicles, being in the composite. In addition, the agent can also include another active ingredient capable of improving the melanin level in melanocytes. The agent can be applied to a subject in need thereof. As an example, the agent can be applied to the skin (or the white hair) of the subject in an effective dosage of 200-400 mg/cm². The vehicle can be an excipient or an additive, and can be further manufactured into a solution type, a lotion type, a cream type or a gel type that is easy to take.

To evaluate the extract of cocoa rind can improve the melanin level in melanocytes, 100 grams of the sample of cocoa rind are mixed with 200 mL of the aqueous ethanol solution with the concentration of ethanol being 75%), followed by extracting at 70° C. for 7 hours. The supernatant obtained by centrifugation (10,000 rpm for about 40 minutes) is filtered, concentrated under reduced pressure and freeze-drying to form the extract of cocoa rind. The B16-F10 skin melanoma cells are used as the model cell line.

Trial (A).

Referring to TABLE 1, the extract of cocoa rind is added in the B16-F10 cells of groups A1-A3 for 24 hours. Survival rates of the B16-F10 cells of groups A0-A3 are calculated by MTT method.

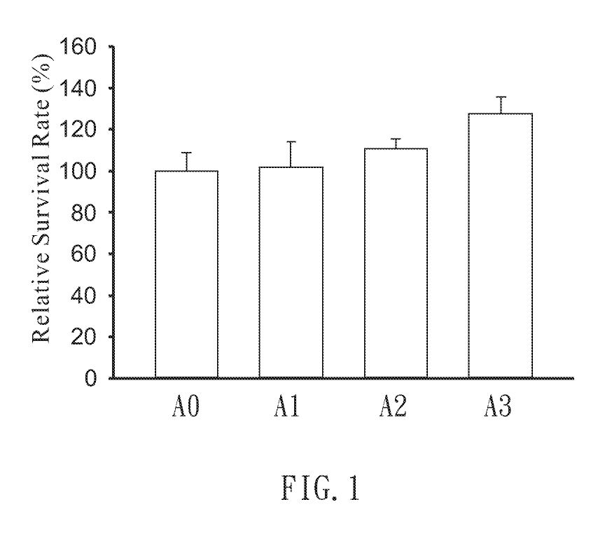

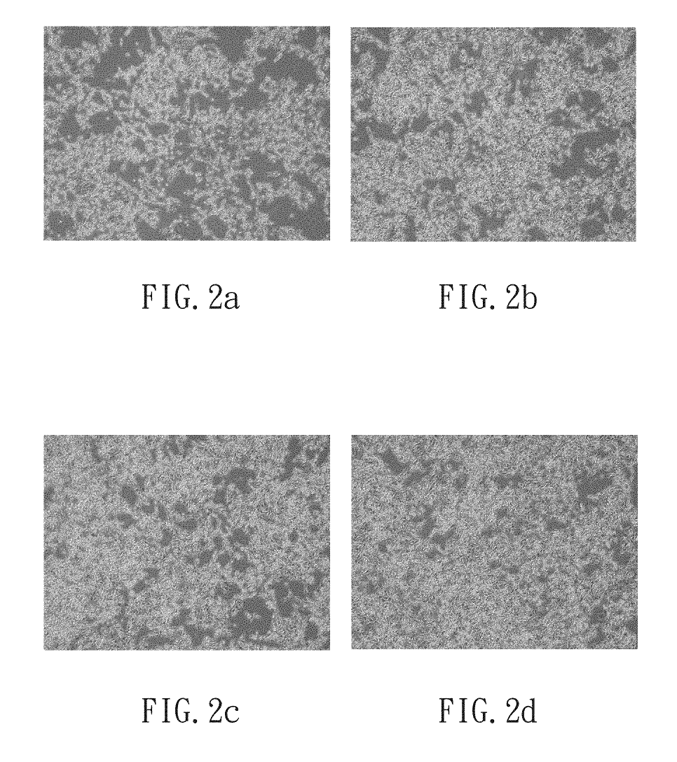

TABLE 1

| | Extract of cocoa rind | |
|---|---|---|
| Group | (μg/mL) | FIGS. |
| A0 | 0 | FIGS. 1 and 2a |
| A1 | 100 | FIGS. 1 and 2b |
| A2 | 200 | FIGS. 1 and 2c |
| A3 | 400 | FIGS. 1 and 2d |

Referring to FIGS. 1 and 2a-2d, the addition of the extract of cocoa rind can improve the survival of melanocytes.

Trial (B).

Referring to TABLE 2, the extract of cocoa rind is added in the B16-F10 cells of groups B1-B3 for 24 hours. Melanin in the B16-F10 cells of groups B0-B3 is dissolved by sodium hydroxide (NaOH). Melanin levels of the B16-F10 cells of groups B0-B3 are calculated according to the absorbance at 405 nm.

TABLE 2

| Group | Extract of cocoa rind (μg/mL) |
|---|---|
| B0 | 0 |
| B1 | 100 |
| B2 | 200 |
| B3 | 400 |

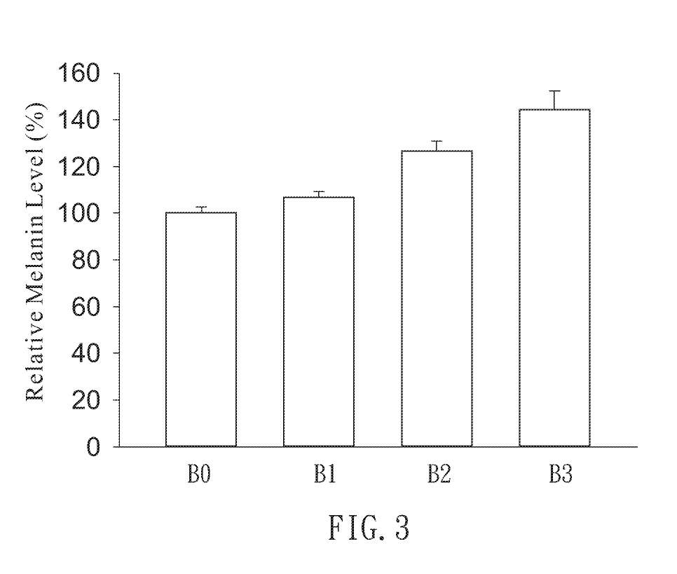

Referring to FIG. 3, the addition of the extract of cocoa rind can increase the melanin level in melanocytes. The melanin level of group B3 (with the concentration of the extract of cocoa rind being 400 μg/mL) increases up to 37%.

Trial (C).

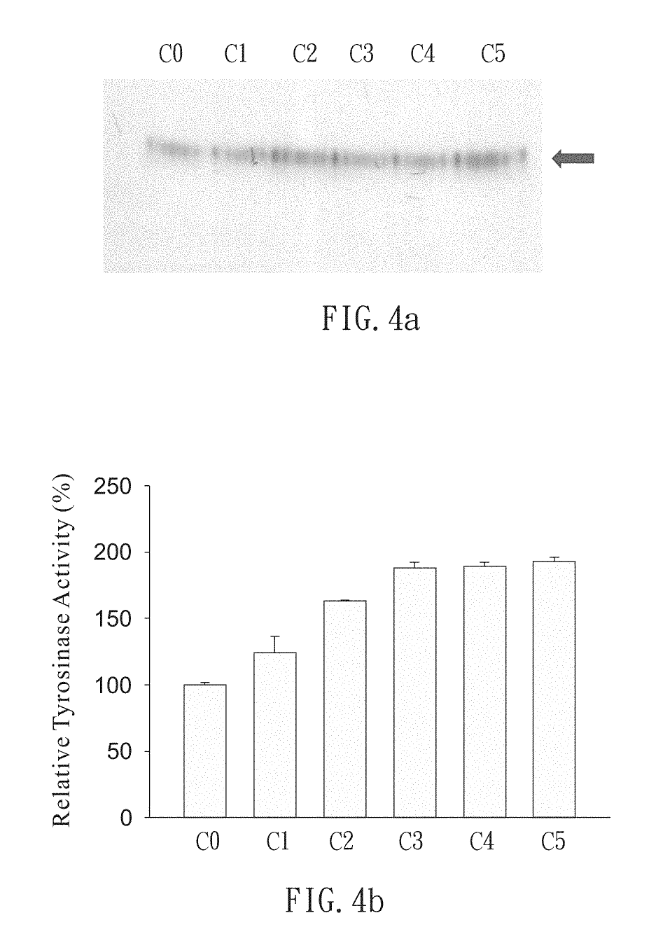

Referring to TABLE 3, the extract of cocoa rind is added in the B16-F10 cells of groups C1-05 for 24 hours. Gel with electrophoretic separated tyrosinase is placed in a buffer including tyrosine and dopamine. The tyrosinase activities of B16-F10 cells of groups C0-05 are calculated according to the band intensities on the gel shown in FIG. 4a.

TABLE 3

| Groups | Extract of cocoa rind (μg/mL) |
|---|---|
| C0 | 0 |
| C1 | 25 |
| C2 | 50 |
| C3 | 100 |
| C4 | 200 |
| C5 | 400 |

Referring to FIG. 4a and FIG. 4b, which is a quantitative bar chart according to FIG. 4a, the addition of the extract of cocoa rind can improve tyrosinase activity.

Trial (D).

Referring to TABLE 4, the extract of cocoa rind is added in the B16-F10 cells of groups D1-D3 for 24 hours. Lysates of the B16-F10 cells of groups D0-D3 are collected. Levels of proteins associated with melanin synthesis, such as tyrosinase, TRP-1 and DCT, are detected by Western blot.

TABLE 4

| Groups | Extract of cocoa rind (μg/mL) |
|---|---|
| D0 | 0 |
| D1 | 100 |
| D2 | 200 |
| D3 | 400 |

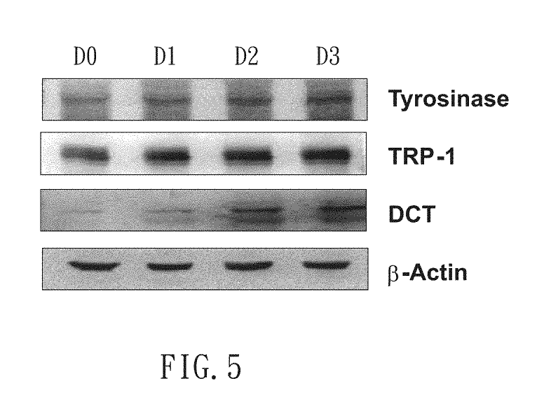

Referring to FIG. 5, the addition of the extract of cocoa rind can improve levels of proteins associated with melanin synthesis, such as tyrosinase, TRP-1 and DCT.

Accordingly, the active ingredients extracted from the sample of cocoa rind can effectively increase the expression level and the activity of tyrosinase in melanocytes, improving the melanin level in melanocytes. Therefore, the extract of cocoa rind can be used to applied for manufacturing a tanning agent or a black hair agent. As such, the cocoa rind, which is usually thought as the waste of manufacturing chocolate, has a new economic output.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A method for sunless tanning, comprising applying an effective amount of an extract of cocoa rind to a skin of a subject in need thereof, wherein the extract of cocoa rind is obtained by extracting a sample of cocoa rind by an aqueous ethanol solution with a concentration of ethanol being 50-75%.

2. The method for sunless tanning as claimed in claim 1, wherein the extract of cocoa rind is applied in the effective amount being 200-400 mg/cm².

3. A method for darkening white hair, comprising applying an effective amount of an extract of cocoa rind to a white hair of a subject in need thereof, wherein the extract of cocoa rind is obtained by extracting a sample of cocoa rind by an aqueous ethanol solution with a concentration of ethanol being 50-75%.

4. The method for darkening white hair as claimed in claim 3, wherein the extract of cocoa rind is applied in the effective amount being 200-400 mg/cm².
